(19) United States
(12) Patent Application Publication
(10) Pub. No.: US 2008/0275299 A1
(43) Pub. Date: Nov. 6, 2008

(54) ACTUATION DEVICE

(75) Inventor: Chul Hi Park, Los Altos, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Chul Hi Park, Los Altos, CA (US)

(21) Appl. No.: 12/113,073

(22) Filed: Apr. 30, 2008

Related U.S. Application Data
(60) Provisional application No. 60/915,423, filed on May 1, 2007.

(51) Int. Cl. A61B 1/01

(52) U.S. Cl. 600/115

(57) ABSTRACT

An inflatable actuation device that advantageously utilizes fluid pressure to actuate a portion of the shaft of a flexible elongated instrument for performing medical and industrial procedure in a tortuous passage and method of use are described herein. The inflatable actuation device allows an operator to actively manipulate a portion of the elongated instrument shaft within the passage, which facilitate the insertion of the instrument, for example, a medical endoscope into the passage. The present inflatable actuation device comprises an annular bladder, which can be expanded and collapsed, with a lumen to receive the elongated instrument. The inflatable actuation device is activated by inflating the annular bladder with pressurizing fluid supplied from a pressurizing fluid supply controller outside the passage.

### CROSS REFERENCE TO RELATED APPLICATIONS

The present application claims priority to U.S. Provisional Application No. 60/915,423, filed on May 1, 2007, which is incorporated by reference.

### BACKGROUND OF THE INVENTION

The present invention relates generally to an actuation device and a method of use thereof. More particularly, it relates to an actuation device that is operated, in part, by fluid pressure and a method of using the actuation device to facilitate insertion of a flexible, elongated instrument (such as a medical endoscope) into a tortuous passage and maneuver the instrument therein for diagnostics, examination and/or treatment in medical and industrial applications.

A flexible endoscope is a medical instrument having an elongated tubular shape for viewing the interior of a patient's body. Flexible endoscopes can be used for a variety of different diagnostic and interventional procedures, including colonoscopy, sigmoidoscopy, bronchoscopy, thoracoscopy, laparoscopy and video endoscopy.

Colonoscopy is a medical procedure in which a flexible endoscope, or colonoscope, is inserted into patient's colon for diagnostic examination and/or surgical treatment of the colon. A standard colonoscope is typically 135-185 cm in length and 12-19 mm in diameter. It may include a fiber-optic imaging bundle or a miniature camera located at the instrument's tip, illumination fibers, one or two instrument channels that may also be used for insufflation or irrigation, air and water channels, and vacuum channels. A colonoscope is sufficiently stiff so that it does not buckle when it is pushed from outside the patient during insertion, yet flexible enough to be maneuvered through the tortuous lumen of the colon. The final six inches of the distal end of a colonoscope can be controlled by an endoscopist from outside the patient.

The most commonly used procedure for examining the colon is to insert a colonoscope as far into the colon as desired while inspecting as the colonoscope is advanced. A detailed examination of the colon is made as the colonoscope is withdrawn. To examine the entire colon, the colonoscope is inserted through the anus into the rectum, and then advanced through the sigmoid colon into the descending colon. The colonoscope then passes through the left colic flexure (the splenic flexure) into the transverse colon, and then through the right colic flexure (the hepatic flexure). The colonoscope next passes through the ascending colon and finally reaches the cecum.

To insert and advance a colonoscope into the colon, an endoscopist employs a number of elaborate maneuvers to negotiate numerous bends and turns normally found in the colon. The portion of a colonoscope shaft that can be maneuvered by an endoscopist is about 15 cm of the distal tip of the instrument, called the bending section, that can be tilted by up to 180 degrees in four directions (i.e., up and down, left and right). To advance a colonoscope, an endoscopist grasps the instrument at a point outside the body near the anus and pushes it inwards while maneuvering the bending section so that the tip of the instrument is pointed toward the direction of open lumen. Often the direction the tip of the instrument is pointing is not aligned with the axis (or direction) of the shaft just behind the bending section. When an endoscopist advances the instrument expecting the instrument to head in the direction the tip is pointed to, it frequently moves in an unanticipated direction and the tip ends up bumping into the colon wall. The end result of this maneuver is a “red-out,” a phenomenon named after the reddish hue which fills a monitor that displays images from the camera at the tip of the colonoscope. To recover from this misdirection an endoscopist must pull back the instrument, steer the bending tip toward open lumen and try advancing the instrument again. Considerable time and effort are spent in repeating this frustrating maneuver as an endoscopist negotiates numerous bends in the colon in a typical colonoscopy procedure.

Repeated applications of steering and advancing maneuvers described above often leaves a crooked and/or a loop-like formation in the shaft of an endoscope. This formation tends to be exaggerated whenever the advancing portion of the shaft or tip of an endoscope is confronted with an obstacle such as a sharp bend or other form of resistance such as friction between the shaft and the colon wall. If not dealt with, the enlargement of these formations are bound to become a main cause of patient pain and serious difficulties in advancing the instrument deeper into the colon. To undo this undesirable formation in the endoscope shaft, an endoscopist pulls back while twisting the instrument in a counter-clockwise or clockwise direction to remove a crooked or looped configuration in the shaft. This is usually done after the tip of endoscope enters the descending colon or passes the splenic flexure. The portion of the colon that has been entered by the shaft of an endoscope prior to this maneuver gets effectively pleated where the shaft of the endoscope becomes shortened and straightened. This maneuver generally requires a fairly high level of skill on an endoscopist's part and usually causes severe discomfort to the patient. Even for an experienced endoscopist this maneuver is a time consuming process and often must be repeated multiple time before a desired result can be obtained partly because an endoscopist has no means to verify the progress and results of the maneuvers.

The formation of a loop in the shaft of a colonoscope is a common occurrence during intubation of the colon. In some people the sigmoid colon can be very long and is unfixed, except by its mesentery, and so can be extremely difficult to intubate due to its predisposition to form loops when a colonoscope is pushed through it. Some anatomical landmarks, such as rectosigmoidal junction, splenic flexure and hepatic flexure, are difficult to pass through simply because of their tortuous nature with present endoscope technology. Problems traversing these areas are exacerbated by looping of the colonoscope and subsequent stretching of the sigmoid colon causing discomfort for the patient undergoing the procedure.

Oshiro (U.S. Pat. No. 4,040,413), which is incorporated by reference, discloses an endoscope device equipped with a plurality of balloons near the tip end for use in a narrow body cavity. The balloons are primarily used to expand the body cavity so that the manipulation of a bending section of an endoscope can be facilitated for improved visualization of the cavity. There is a suggestion for a method for facilitating the insertion of endoscope into a body cavity with the use of the balloons to enlarge a space in the cavity. The balloons are not used to actively actuate the shaft of endoscope to change its shape or to facilitate insertion into a body cavity.

Maki, et. al. (U.S. Pat. No. 6,478,772), which is incorporated by reference, disclose a method of inducing bending in a medical tube. The method involves a balloon device with an envelope of non-uniform stretching characteristics causing the balloon to take on an asymmetrical shape when inflated with respect to the medical tubing it is mounted on. The actuation characteristics that determine the degree and geometry of bending depend on the design configuration and stretching characteristics of the balloon, which limits the applicability of the device and method disclosed.

Ortiz (U.S. Pat. App. No. 20070106302), which is incorporated by reference, discloses lumen traversing devices comprising an elongated body attached with a pair of radially expandable tubing elements. Advancing motion of the elongated body is achieved by moving the tubing elements along a body lumen while alternately expanding and contracting them. The tubing elements are used to provide traction against the lumen wall but not as a means for actuating the elongated body. The use of this device and method requires cumbersome operation requiring both hands of an operator.

U.S. patent application Ser. No. 11/872,025, assigned to this inventor and incorporated by reference, discloses an inflatable actuation device partly comprising an inflatable bladder with a lumen and a sleeve in which the inflatable bladder is disposed. The inflatable bladder and sleeve are configured such that only a circumferential portion of the inflatable bladder, when fully inflated, comes in contact with and applies force to the sleeve, which urges the sleeve to return to a design configuration. Even when the bladder is in a fully inflated state there remain open spaces between the bladder and the sleeve. The force acting on the sleeve is transferred to and actuates the endoscope shaft to effect a straightening or a bending.

There is a need to provide an improved device and method for inserting an instrument into a living organ having a tortuous passage for medical diagnostics, examination and/or treatment.

### BRIEF SUMMARY OF THE INVENTION

In order to provide a clear and consistent understanding of the specification and claims, the following definitions are provided.

For the purposes of this disclosure, including the appended claims, the terms “distal”, “distally”, and “distal end”, as they relate to the devices and methods described herein, refer to the end of the device further from or in the direction away from an operator who might be applying the device or method to the subject. Stated otherwise, the terms refer to the end of the device closer to or in the direction towards the patient's interior.

The terms “proximal”, “proximally”, and “proximal end”, as they relate to the devices and methods described herein, refer to the end of the device closer to or in the direction towards the operator who might be applying the device or method, rather than the patient.

As used herein, the terms “passage”, “bodily passage”, and “tortuous passage” are interchangeably used to refer to passages existing in human or animal anatomy such as the colon or a passage found in an industrial setting.

The terms “actuation device” and “inflatable actuation device” are used interchangeably, hereinafter.

In one embodiment, the present actuation device generally comprises a substantially annular bladder with a lumen therein consisting of an inner sleeve bounding the lumen and a shaped, outer sleeve and a supply tube. The substantially annular bladder receives a flexible, elongated instrument in the lumen and actuates it to a predetermined configuration when inflated by pressurizing fluid supplied through the supply tube from a source external to the passage where the elongated instrument is introduced. Although the embodiments described in this specification specifically refers to the colon and colonoscopy procedure, the scope of their applicability is not limited to any particular bodily organ, other non-bodily passage or procedure.

In one aspect of this invention, the substantially annular bladder comprising the present actuation device may be configured to have a plurality of toroidal or doughnut-shaped compartments that are substantially symmetrical with respect to the bladder axis and in fluid communications with one another. In another aspect, the toroidal compartments may be of asymmetrical shapes. In yet another aspect, a flexible sleeve tightly encircling the toroidal compartments in a fully inflated state may be employed to further provide columnar strengthen to the substantially annular bladder.

In another aspect of this invention, the toroidal compartments may be further divided circumferentially into a plurality of axial compartments, which are in fluid communications with one another, with inter-compartment gaps running substantially parallel to the bladder axis. The gaps between axial compartments in different toroidal compartments may be disposed substantially aligned with one another. Alternately, they may be disposed staggered with respect to one another in a predetermined manner.

In further aspect of this invention, all toroidal compartments comprising the annular bladder may be further divided circumferentially into an equal number of mutually isolated axial compartments with gaps between axial compartments disposed substantially parallel to the axis of the annular bladder and aligned with those in immediately adjacent toroidal compartments. A linear chain of axial compartments from different toroidal compartment are configured interconnected and in fluid communications with one another. Each of the linear chains of interconnected axial compartments is connected to a separate supply of pressurizing fluid and can be inflated or deflated independently from one another.

Alternately, axial compartments from different toroidal compartments may be grouped and interconnected in a predetermined, more complex pattern than a line. Each of these groups of interconnected axial compartments is connected to a separate supply of pressurizing fluid and can be inflated or deflated independently.

In one aspect of the present invention, a method of moving along a passage a flexible, elongated instrument fitted with the actuation device comprising an annular bladder with a plurality of symmetrical toroidal compartments comprises; inserting said flexible, elongated instrument within the passage; and inflating said annular bladder to straighten a portion of said flexible, elongated instrument.

In another aspect of the present invention, a method of moving along a passage a flexible, elongated instrument fitted with the actuation device comprising an annular bladder with a plurality of toroidal compartments divided into a plurality of independent chains of interconnected axial compartments grouped into predetermined patterns around the axis of said annular bladder comprises; inserting said flexible, elongated instrument within the passage; inflating a chain of interconnected axial compartments with other selected chains in a deflated or inflated state to put a portion of the elongated instrument into a curved shape; and inflating the chain in opposing positional relationship from said chain with respect to the elongated instrument to straighten the curved portion of the elongated instrument and urge the distal tip to advance.

In another aspect of the present invention, a method of navigating a tortuous passage a flexible, elongated instrument fitted near its distal end with the actuation device comprising an annular bladder with a plurality of toroidal compartments divided into a plurality of linear and independent chains of interconnected axial compartments aligned along the axis of said annular bladder comprises; inserting said flexible, elongated instrument within the passage; inflating a linear chain of compartments with other chains in a deflated state to effect a deflection of the distal portion of the elongated instrument; and deflating said chain of axial compartments and inflating the chain diagonally located from said chain with respect to the elongated instrument to effect a deflection of the distal portion of the elongated instrument in opposite direction to unravel a bend in the colon and open up a closed lumen.

### BRIEF DESCRIPTION OF THE DRAWINGS

The following exemplary figures are provided to supplement the description below and more clearly describe the invention. In the figures, like elements are generally designated with the same reference numeral for illustrative convenience and should not be used to limit the scope of the present invention.

FIG. 1A is a schematic, perspective view of a portion of an annular bladder in an inflated state and mounted on an endoscope shaft according to an embodiment of the present invention.

FIG. 1B is a sectional view taken along the line 1B-1B of an annular bladder in an inflated state, shown in FIG. 1A, according to an embodiment of the present invention.

FIG. 1C is a sectional view, similar to FIG. 1B, along the axis of an annular bladder in an inflated state according to another embodiment of the present invention.

FIG. 1D is a sectional view, similar to FIG. 1B, along the axis of an annular bladder encircled by a sleeve in an inflated state according to yet another embodiment of the present invention.

FIG. 1E is a sectional view, similar to FIG. 1D, along the axis of an annular bladder encircled by a fitted sleeve in an inflated state according to yet another embodiment of the present invention.

FIG. 1F is a perspective view of a fitted sleeve as shown in FIG. 1D in a fully expanded state according to an embodiment of the present invention.

FIG. 1G is a sectional view, similar to FIG. 1B, along the axis of a portion of an annular bladder having a plurality of intervening gap portions among banks of toroidal compartments according to yet another embodiment of the present invention.

FIG. 2 is a sectional view along the axis of a portion of an annular bladder in an inflated state with asymmetrical toroidal compartments according to an embodiment of the present invention.

FIG. 3A is a schematic, perspective view of a toroidal compartment comprising the annular bladder divided into a plurality of axial compartments according to an embodiment of the present invention.

FIG. 3B is a sectional view taken along the line 3B-3B of a toroidal compartment in an inflated state, shown in FIG. 3A, according to an embodiment of the present invention.

FIG. 4 is a schematic, perspective view of a portion of an annular bladder in an inflated state with axial compartments from different toroidal compartments aligned with one another according to an embodiment of the present invention.

FIG. 5 shows an illustrative example of a mode of activation of the actuation device for bending a portion of the instrument shaft according to an embodiment of the present invention.

FIG. 6 is a schematic, perspective view of a portion of an annular bladder in an inflated state with axial compartments from different toroidal compartments grouped in a predetermined pattern according to an embodiment of the present invention.

FIG. 7 shows an illustrative example of a mode of activation of the actuation device for introducing a curved shape to a portion of the instrument shaft according to an embodiment of the present invention.

FIG. 8 is a schematic representation showing the effect of straightening a portion of the elongated instrument in a curved configuration according to an embodiment of the present invention.

FIG. 9 is a schematic representation showing steps for advancing the elongated instrument according to an embodiment of the present invention.

FIG. 10 is a schematic representation showing a wide angle sweeping motion of the distal portion of the elongated instrument according to an embodiment of the present invention.

### DETAILED DESCRIPTION OF THE INVENTION

Embodiments of the present invention relate to an actuation device that utilizes fluid pressure to assist in the insertion of a flexible, elongated instrument, for example, a medical flexible endoscope into a tortuous passage and maneuver therein, and method of use.

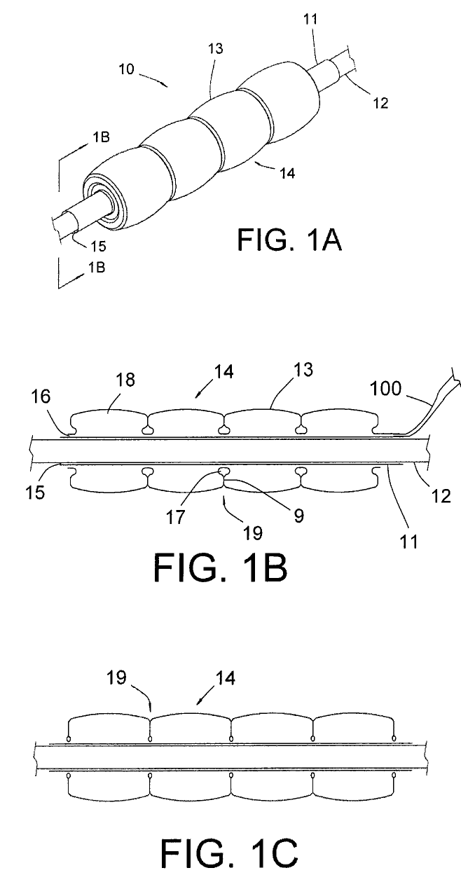

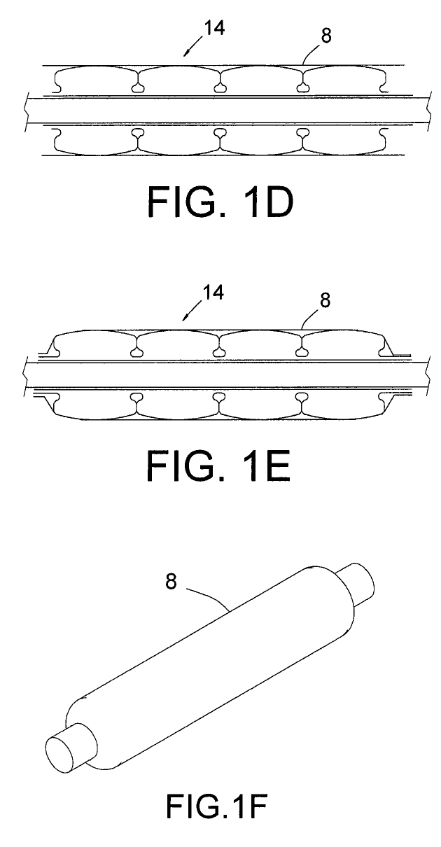

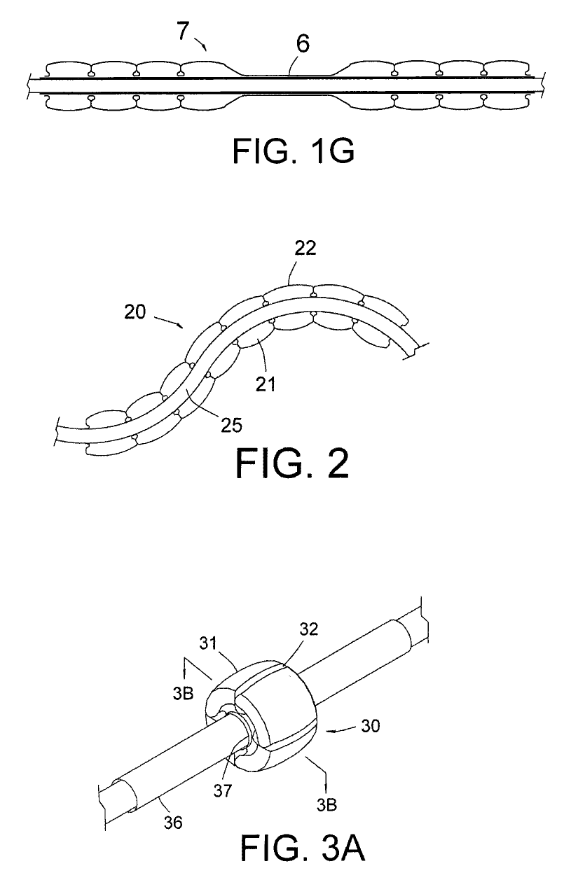

Referring to FIG. 1A, a portion of the annular bladder 14 comprising the actuation device 10 in an inflated state is schematically shown mounted on the shaft of a flexible endoscope 12. Actuation device 10 generally comprises a substantially annular bladder 14 with a lumen (or passage) 15 including an inner sleeve 11 bounding lumen 15, a shaped, outer sleeve 13 and a supply tube (not shown). Inner 11 and outer 13 sleeves are sealingly joined circumferentially in proximal and distal locations to form fluid tight annular bladder 14. One or more supply tubes 100 are provided to supply pressurizing fluid to the annular bladder from a pressurizing fluid supply controller outside the passage. In an embodiment, the supply tube may be embedded in or integrated with inner sleeve 11 with a plurality of outlets disposed at predetermined locations to provide the pressurized fluid between the inner and outer sleeves 11 and 13.

FIG. 1B shows a cross section taken along the line 1B-1B in FIG. 1A. Outer sleeve 13 is configured to divide annular bladder 14 along its axis into a plurality of toroidal compartments 18 of substantially symmetrical shapes with respect to the axis of annular bladder 14 separated by annular gaps 19 in one aspect of the present invention. Toroidal compartments 18 are not symmetrical in shape in other embodiments. Adjacent toroidal compartments 18 are in fluid communication with each other through annular openings 16. Openings 16 are annular in the present embodiment but are not limited to such. In one embodiment, inner and outer sleeves 11 and 13 may be joined or bonded together at several predetermined locations along the inner circumference of annular gaps 19 and across annular openings 16 without completely closing annular openings 16 to keep adjacent toroidal compartments in fluid communication with each other.

The inner diameter of the inner sleeve may be chosen to be large enough to receive an elongated instrument such as a medical endoscope (e.g., between 5 mm and 15 mm). In one embodiment, the outer diameter of the shaped outer sleeve or the annular bladder is between 20 mm and 50 mm and the diameter of the shaped outer sleeve at the bottom of the annular gap is between 6 mm and 16 mm. The axial length of a toroidal compartment may be optimized in consideration of the mechanical characteristics of the endoscope and those of the passage where the endoscope is deployed. In an embodiment, this length is between 2 cm and 15 cm, preferably, between 5 cm and 10 cm. The thicknesses of the inner sleeve and the shaped outer sleeve may be chosen as thin as practically possible to be able to resist bursting under the pressure inside the bladder. The thicknesses may be between 0.01 mm and 0.05 mm. The inner and outer shaped sleeves may be made of a material and thickness that allow them to be readily folded into a low profile form to facilitate insertion of an actuation device mounted on an endoscope into a passage through a constricted opening.

The construction materials for the inner and shaped outer sleeves may be flexible and substantially non-compliant or semi-compliant. A non-compliant material resists stretching and maintains a design length or a design shape even when acted on by a force not exceeding a given magnitude. A bladder comprising sleeves of non-compliant materials is able to withstand internal pressure in a fully inflated state and maintain its design shape without expanding freely in proportion to the internal pressure, as a latex balloon would, until it ruptures. These materials may be soft enough to allow the annular bladders in a deflated state to be pleated into a low profile form. Suitable materials includes thermoplastics such as polyethylene terephthalate (PET), polypropylene, polyamide (Nylon), polyimide (Kapton), polyvinylchloride (PVC), polyurethane and polyethylene of various grades. Alternately, latex or silicon rubber material may be employed for the construction. Any number of methods for joining or bonding inner and outer sleeves may be employed. For example, a thermal bonding or a bonding method based on an adhesive, or a combination of both may be utilized. An intermediate polymer layer such as ethylene-vinyl acetate (EVA) may be used for a thermal bonding to enhance the bonding strength and to facilitate the bonding. A predetermined mechanical joining method may also be employed to provide bonding as well as means for relieving strain on the joint. A blow molding, a thermo-forming or a number of other methods well know to the art of shaping a thin film polymeric material may be employed to construct the shaped outer sleeve. Any low viscosity fluid may be used to inflate the annular bladder, for example, air, carbon dioxide, water and a saline solution.

In an aspect of this invention, outer sleeve 13 is configured such that the volumes occupied by adjacent toroidal compartments 18 in a fully expanded state partially overlap all around annular gap 19 when the endoscope shaft and the present actuation device are in a straightened configuration. In this configuration at least a portion of envelopes of adjacent toroidal compartments 18 along annular gap 19 always remains in contact with each other even when the actuation device is partially inflated. The shape of toroidal compartment 18 is such that portions of outer shaped sleeve 13 comprising envelopes of the adjacent toroidal compartments come in contact with each other, as the actuation device is gradually inflated, along outside portion 9 of annular gap 19. Even after the actuation device reaches a fully inflated state, there can remain an open portion 17 in annular gap 19 between neighboring annular compartments 18. Alternately, annular gap 19 may be allowed to close substantially completely, as schematically depicted in FIG. 1C in an illustrative example.

Adjacent toroidal compartments apply forces of equal magnitude, when activated by internal pressure, against each other through intervening gaps during expansion and after the actuation device is fully inflated. It is well known to those familiar with the art of inflatable structures that a cylindrical inflatable vessel under pressure exhibits certain degree of structural strengths (for example, see J. D. Suhey, N. H. Kim, C. Neizrecki, “Numerical modeling and design of inflatable structures-application to open-ocean-aquaculture cages.”, Aquacultural Engineering 33 (2005), 285-303, which is incorporated by reference). The toroidal compartment with a given internal pressure supports a portion of the shaft of a flexible endoscope encircled therein and causes it to behave as a substantially rigid and straight segment. A net force exerted between adjacent toroidal compartments drives the change in the relative orientation of two shaft segments with respect to the intervening gap by causing bending or deflection of a portion of the endoscope shaft spanned by the two toroidal compartments. This actuation force diminishes gradually as the bent portion of endoscope shaft becomes straightened and the toroidal compartments return to their symmetrical design configuration. The actuation also stops when the net contact force between neighboring annular bladders is balanced by an external load exerted on the portion of endoscope shaft spanned by them. The magnitude of actuation force may be continuously varied by adjusting the internal pressure. For example, when the actuation device of the present invention is used to manipulate the shaft of colonoscope inside the colon the magnitude of actuation force may be between 2 Newton (or N) to 35 N, preferably, between 5 N and 20 N, which would be sufficient to displace the colon and its surroundings. The operating pressure of present actuation device may be between 1 atm and 10 atm, preferably, between 1.5 atm and 5 atm.

In another aspect of this invention, there is provided a flexible sleeve 8, as schematically depicted in FIG. 1D, disposed to tightly encircle annular bladder 14 around the circumference thereof when it is fully inflated to increase the columnar strength of annular bladder 14 and to provide additional actuation force for straightening or unbending the endoscope shaft. It is well known to those familiar with the art of inflatable structures that a non-compliant sleeve exhibits a restoring force in the form of tensile stress when expanded by a force when it is in a collapsed configuration or deformed out of a design configuration. This restoring force generally acts in the direction that brings the deformed sleeve back to its design configuration. In an embodiment of the present invention, the force acting on flexible sleeve 8 is provided by the internal pressure in annular bladder 14. The restoring force on flexible sleeve 8 complements the actuation force produced by the annular bladder.

FIG. 1E shows a cross sectional view along the axis of the annular bladder of an alternate configuration of flexible sleeve 8 that is tightly fitted to the outer contour of the annular bladder in a fully inflated state around the circumference as well as both ends thereof, which is shown in perspective view in FIG. 1F. Although the sleeve shown in these drawings are generally in a cylindrical shape, it is to be appreciated the sleeve may be of any shape to fit tightly around the annular bladder in a chosen application of the actuation device of the present invention. It is well known to those familiar with the art of inflatable structures that a sleeve of a geometry with both ends partially closed in, such as shown in FIG. 1F, is able to provide stronger restoring force, when acted on by internal pressure, than a sleeve of a geometry with both ends open, such as that shown in FIG. 1E. A sleeve with closed ends experiences not only a stretching force in hoop or radial direction as a sleeve with open ends does when acted on by an internal pressure but along the axis as well because of the pressure force acting on either ends in opposite directions along the axis of the sleeve.

The construction material for flexible sleeve 8 may be flexible and substantially non-compliant. This material preferably should be soft enough to allow flexible sleeve 8 to be pleated into a low profile form when not stretched by the annular bladder. Materials suitable for the sleeve are those used to construct the inner and outer sleeves of the annular bladder. In a further aspect, as shown in FIG. 1G, annular bladder 7 may include a plurality of intervening open portions 6 devoid of toroidal compartments at predetermined locations among banks of densely disposed toroidal compartments.

As in FIG. 2, schematically showing a portion of the annular bladder comprising the actuation device in a fully inflated state in an embodiment of the present invention, shaped outer sleeve 22 of annular bladder 20 may be configured to make toroidal compartments 21 asymmetrical with respect to the general axis of annular bladder 20. In one embodiment, toroidal compartment 21 may be configured to be thicker on one side of lumen 25 than the opposite side. In this configuration the actuation device comprising annular bladder 20 takes on a curved shape when fully inflated.

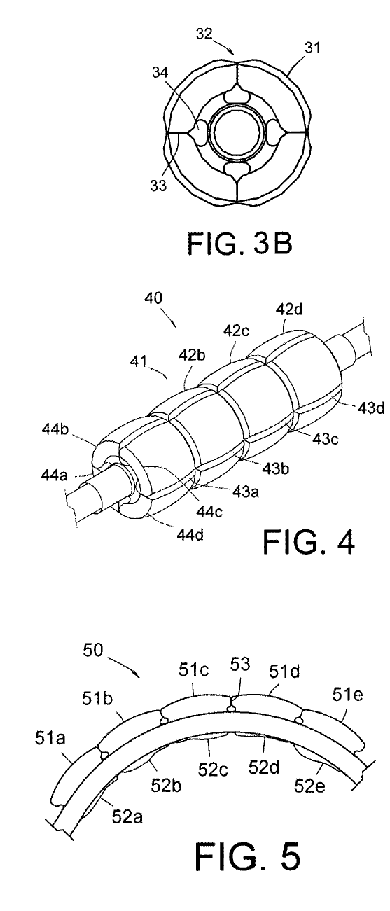

In one embodiment, at least one toroidal compartment of the annular bladder may be further divided into a plurality of axial compartments 31 that are in fluid communication with one another as schematically depicted in FIG. 3A where only one toroidal compartment 30 is shown for clarity. The gaps 32 between adjacent axial compartments 31 may be configured to be substantially parallel to the general axis of the annular bladder. Inner and outer sleeves 36 and 37 may be joined or bonded together at several predetermined locations across the opening along the bottom of gap 32 while keeping adjacent axial compartments in fluid communication with each another. Referring to FIG. 3B, which is a sectional view taken along the line 3B-3B in FIG. 3A, in one aspect of the present invention axial compartments 31 are configured such that the envelopes of adjacent axial compartments first come in contact with each other in gaps 32 near peripheries 33 as the annular bladder is inflated. Even when the annular bladder reaches a fully inflated state there can remain an open portion 34 in gaps 32. Alternately, gaps 32 may be allowed to close substantially completely when the annular bladder is fully inflated. The number of axial compartments in a toroidal compartment may be between 2 and 8, preferably, between 4 and 6.

Gaps between axial compartments comprising a toroidal compartment generally adds mechanical strength to the toroidal compartment, especially columnar strength. In another embodiment, a fitted sleeve (not shown) may be further disposed to tightly encircle the annular bladder when it is fully inflated to increase the columnar strength of the actuation device.

Referring to FIG. 4 where a portion of an annular bladder comprising the actuation device is schematically depicted, the annular bladder 40 may be configured to comprise a plurality of substantially identical toroidal compartments 41 consisting of equal number of axial compartments 44b, 42b-42d with gaps 43a-43d disposed substantially parallel to the axis of annular bladder 40. Axial compartments 44a-44d in a toroidal compartment 41 are configured completely isolated and independent from one another. Axial compartments 44b, 42b-42d from different toroidal compartments aligned along a line parallel to the axis of annular bladder 40 are in fluid communication with one another as are those in other linear chains of interconnected axial compartments. Each of these linear chains is connected to a supply (not shown) for pressurizing fluid and can be inflated or deflated independently from other linear chains of compartments. There are provided at least one supply tube (not shown) for each of these linear chains. Alternately, supply tubes may be embedded in or integrated with the inner sleeve comprising the annular bladder with outlets disposed at predetermined locations. Although only four toroidal compartments with four axial compartments each are shown in this illustrative example, it is to be appreciated that any number of toroidal compartments with any number of axial compartments may be employed to suit the need of a particular application of the present actuation device.

Referring to FIG. 5, a bending or deflection of a portion of the shaft of an elongated instrument can be induced by activating a selected chain of axial compartments 51a-51e comprising the actuation device 50 by inflating them with pressurizing fluid while leaving diagonally opposite chain 52a-52e and, optionally, other chains in a deflated state. Net forces acting across contact surfaces 53 between adjacent axial compartments in the activated chain 51a-51e cause actuation device 50 and the instrument shaft (not shown) to bend in a direction away from the chain 51a-51e. Bending in the any other direction can be accomplished in a similar manner. By pressurizing a chain of axial compartments 52a-52e, diagonally opposite to the activated chain, to the same pressure in the activated chain, the bent portion of the instrument shaft can be restored to a straight configuration. A sweeping motion of a distal or any other portion of the shaft of an elongated instrument can be accomplished by repeatedly applying the bending actuations in alternate directions using the actuation device mounted on the shaft portion. It is to be appreciated any number of axial compartments may be included in a chain and any number of chains may comprise an annular bladder of the actuation device.

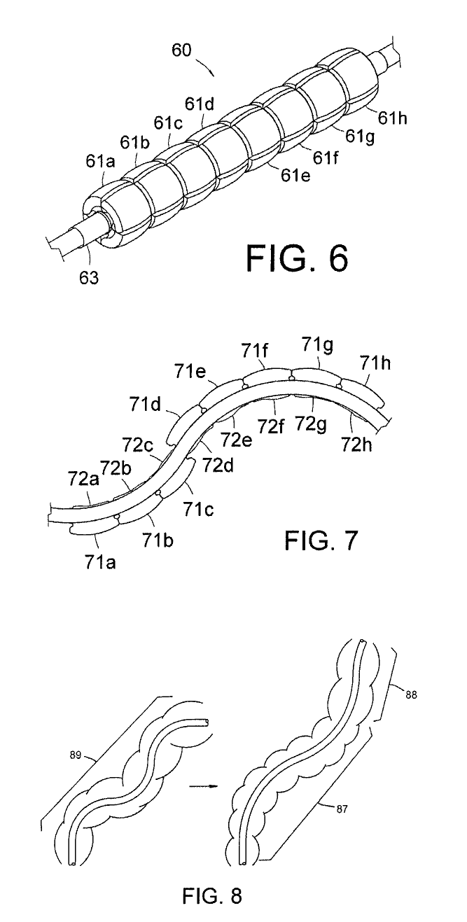

Alternately, as shown in FIG. 6 in a portion of an annular bladder 60 with a plurality of toroidal compartments comprising a predetermined number of mutually isolated axial compartments in an embodiment of the present invention, several groups 61a-61d, 61e-61h of axial compartments from different toroidal compartments may be interconnected in a predetermined pattern around the axis of annular bladder 60. Each of these groups of interconnected axial compartments is connected to a supply for pressurizing fluid and can be inflated or deflated independently. For example, a plurality of independent fluid supply conduits (not shown) may be built into the inner sleeve 63, of which each supplies pressurizing fluid from a pressurizing fluid supply controller outside the passage to a corresponding group of interconnected axial compartments. Although only simple linear patterns are used in grouping axial compartments in this illustrative example, it should be appreciated that more complex pattern of groups of axial compartments may be employed to suit any particular application.

With the bladder configuration described immediately above, a predetermined curved shape may be introduced to a portion of the shaft of an elongated instrument as schematically illustrated in FIG. 7 in an embodiment of the present invention. To introduce an S shape to a portion of the shaft of an elongated instrument, a group of axial compartments 71a-71h may be activated by inflating them with pressurizing fluid while leaving diagonally opposite chain 72a-72h and, optionally, other chains in the annular bladder in a deflated state. It is to be appreciated that other shapes may be introduced to the shaft of an elongated instrument in a similar manner using appropriate grouping patterns of axial compartments. By pressurizing a chain of axial compartments 72a-72h diagonally opposite to the activated chain to the same pressure in the activated chain, the bent portion of the instrument shaft can be restored to a straight configuration.

In one aspect of the present invention, a method of advancing along a tortuous passage a flexible elongated instrument fitted with the actuation device comprising an annular bladder with a plurality of symmetrical, toroidal compartments is described. When the actuation device is activated by inflating with a pressurizing fluid, the net force acting between neighboring toroidal compartments acts on the instrument shaft until the annular bladder returns to a fully inflated and symmetrical state (for example, see FIG. 1B) or the net force between compartments is balanced by an external load applied to the elongated instrument. Similar effects can be achieved with an actuation device with an annular bladder comprising of a plurality of substantially symmetrical toroidal compartments divided into a plurality of axial compartments and by inflating to an equal internal pressure all axial compartments. In an actuation device comprising an annular bladder and a fitted sleeve tightly encircling the annular bladder, tensile stress is generated on the fitted sleeve by the pressure force provided by the annular bladder therein, which always works to restore the sleeve into an initial design configuration, and complements the actuation force from the annular bladder. The magnitude of this tensile stress is generally correlated with the degree of deformation from the design configuration of the sleeve.

FIG. 8 schematically illustrates a sequence, indicated by an arrow, in the method of advancing the elongated instrument in the colon with the present actuation device mounted at a predetermined location along its length, in one embodiment of the present invention. The elongated instrument is introduced into the colon with the actuation device in a deflated, low profile state. Due to its flexible nature, the elongated instrument inserted into and navigated through numerous bends in the colon by pushing from outside the anus has a strong tendency to develop curvature, represented by bracket 89, along portions of its body length throughout the insertion process. The most tortuous part of the colon, the recto-sigmoid colon, lies just proximal to the anus. If not straightened, the curvature introduced to a portion of the elongated instrument in this part of the colon is likely to make further advancement and maneuver of the elongated instrument into the deeper reaches of the colon more difficult. At any time during the insertion process, an endoscopist may activate the inflatable actuation device to straighten out the curvatures on the endoscope shaft that were introduced up to that point. Alternately, an operator may advance the flexible elongated instrument and the inflatable actuation device to a predetermined distance before activating the actuation device. In one embodiment, an operator may wait until a resistance is felt during advancement, which is often the result of one or more bends introduced into the shaft of a flexible elongated instrument, to activate the inflatable actuation device and straighten the bends. This step also makes the portion of the colon, represented by bracket 87, spanned by the curved portion of an elongated instrument to be pleated into a shorter length and helps the distal portion of the elongated instrument to advance, as represented by bracket 88. Once the endoscope is straightened, the pushing force applied by an endoscopist is transmitted through the endoscope shaft more efficiently and advancing the instrument becomes easier.

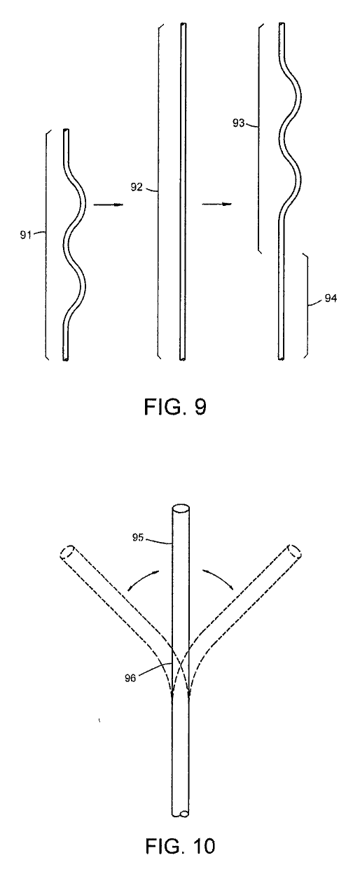

In another aspect of the present invention, a method of moving along a passage a flexible elongated instrument fitted with the present actuation device comprising an annular bladder with a plurality of symmetrical toroidal compartments divided into a plurality of independent groups of interconnected axial compartments is described. As schematically illustrated in FIG. 9 in time sequence, indicated by arrows, during intubation an endoscopist may intentionally introduce curvatures to a portion or several portions of the elongated instrument, depicted by bracket 91 and 93, using one or a combination of methods described previously while pushing a length of the elongated instrument 94 into the colon. The curved portions are then straightened out while keeping the proximal portion of the elongated instrument from sliding out of the colon, which results in advancement of the distal tip of the elongated instrument, as depicted by bracket 92.

In another aspect of the present invention, there is provided a method for assisting the navigation along a passage or the colon with a flexible elongated instrument with the actuation device comprising an annular bladder with a plurality of symmetrical toroidal compartments divided into a plurality of linear and independent chains of interconnected axial compartments aligned along the axis of said annular bladder. In an embodiment of the present invention, the actuation device is mounted close to the distal end of the elongated instrument. A method of inducing a sweeping movement of the straightened portion 95 of the elongated instrument near the distal end is schematically illustrated in FIG. 10 with double headed arrows and the distal end portion depicted with dotted lines. Straightened portion 95 may be the control section of the elongated instrument held in straight configuration or it may additionally include a portion of the elongated instrument proximal to the control section actuated to be in a straight configuration by use of the present actuation device. The sweeping movement can be driven by bending neck portion 96 side to side using the methods described previously. This sweeping movement in addition to manipulation of the control section helps the user to unravel a bend in the colon and find an open lumen as the elongated instrument is advanced along a section of the colon with numerous bends, for example, the recto-sigmoid colon.

While illustrative embodiments of the invention are described above, it will be apparent to those skilled in the art that various changes and modifications may be made therein without departing from the invention. Accordingly, the appended claims should be used to interpret the scope of the present invention.

What is claimed is:

1. An inflatable actuation device for a flexible elongated instrument, the device comprising:
a first sleeve defining a lumen to receive a shaft of the elongated instrument that is configured to be inserted into a tortuous passage; and
a second sleeve, the first and second sleeves defining a bladder configured to be inflated or deflated according to pressure within the bladder; and
at least one supply tube configured to provide pressurizing fluid into the bladder defined by the first and second sleeves;
wherein the first sleeve and the second sleeve are made of material that is substantially non-compliant;
wherein a pressure within the bladder is used to exert a force on the shaft of the elongated instrument and actuate a direction of the shaft.

2. The device of claim 1, wherein the first sleeve is an inner sleeve having an inner surface and outer surface, the inner surface of the first sleeve defining the lumen, and
wherein the second sleeve is an outer sleeve that is provided proximate the outer surface of the first sleeve and away from the lumen.

3. The device of claim 2, wherein said inner sleeve is sealingly joined with the outer sleeve to define the bladder, the bladder including at least one compartment and having an opening coupled to the supply tube to receive the pressurizing fluid.

4. The device of claim 3, further comprising:
a flexible sleeve configured to tightly encircle the bladder when the bladder is in a fully inflated state.

5. The device of claim 4, wherein the flexible sleeve is configured to tightly fit an outside contour of the bladder when the bladder is in a fully inflated state, the bladder having an annular shape.

6. The device of claim 1, wherein the bladder is divided into a plurality of compartments of toroidal and symmetrical configuration with axes thereof aligned parallel to the axis of the bladder.

7. The device of claim 6, wherein the toroidal and symmetrical compartment is further divided into a plurality of chambers arranged circumferentially around the axis thereof.

8. The device of claim 7, wherein the chambers comprising the toroidal compartment are independent from one another and chambers selected from different toroidal compartments are configured to form a geometrical chain of a predetermined configuration and be in fluid communication with one another.

9. The device of claim 8, wherein the chain of chambers is linearly aligned along and at predetermined circumferential positions around the toroidal compartments.

10. The device of claim 8, wherein the chain of chambers is disposed at circumferential positions around the toroidal compartments in a curved geometrical configuration.

11. An inflatable actuation device for a flexible elongated instrument, the device comprising:
a bladder having a flexible envelope configured to be expanded or collapsed, the flexible envelope defining a lumen, the lumen configured to receive a shaft of the elongated instrument that is configured to be inserted into a tortuous passage;
at least one supply tube configured to provide pressurizing fluid into the bladder;
wherein the bladder is made of material that is substantially non-compliant;
wherein a pressure within the bladder is used to exert a force on the shaft of the elongated instrument and actuate a direction of the shaft.

12. The device of claim 11, further comprising:
a flexible sleeve configured to be tightly fitted around an outside contour of the bladder when the bladder is in a fully inflated state,
wherein the shapes of the bladder and the flexible sleeve do not change substantially beyond predetermined shapes when the bladder is inflated to and above a predetermined pressure,
wherein the bladder and the flexible sleeve apply forces to a portion of the shaft of the elongated instrument to bend or straighten the shaft according to a pressure within the bladder.

13. The device of claim 12, wherein the bladder comprises a plurality of compartments in fluid communications with one another.

14. The device of claim 12, wherein the bladder comprises a plurality of compartments independent from one another.

15. A method of advancing a flexible elongated instrument into a tortuous passage, the method comprising:
providing the inflatable actuation device at a predetermined location along a shaft of the elongated instrument;
inserting the elongated instrument and the inflatable actuation device into the tortuous passage;
advancing a distal tip of the elongated instrument along the tortuous passage for a given distance;
pressurizing the inflatable actuation device by supplying fluid through a pressurizing fluid supply tube coupled to the inflatable actuation device; and
straightening the inflatable actuation device and a portion of the elongated instrument.

16. The method of claim 15, further comprising:
repeating the steps of advancing and straightening the inflatable actuation device and the elongated instrument.

17. A method of advancing a flexible elongated instrument into a tortuous passage, the method comprising;
providing an inflatable actuation device at a predetermined location along a shaft of the elongated instrument;
inserting the elongated instrument and the inflatable actuation device into the tortuous passage;
advancing a distal tip of the elongated instrument along the tortuous passage for a given distance by applying a directional force on the elongated instrument from outside the tortuous passage;
activating the inflatable actuation devices by pressurizing the inflatable actuation device with pressurizing fluid to effect bending of the inflatable actuation devices to introduce a curved shape to a portion of the elongated instrument that is inside the tortuous passage while pushing the elongated instrument into the tortuous passage as the elongated instrument is drawn into the tortuous passage in step with the formation of the curved shape; and
activating the inflatable actuation device by pressurizing the inflatable actuation device with pressurizing fluid to effect straightening of the inflatable actuation device to straighten the curved shape of the portion of the elongated instrument that is inside the tortuous passage to promote the advancement of a tip of the elongated instrument,
wherein the elongated instrument is bent and straightened repeatedly as the elongated instrument is made to advance into the tortuous passage.

18. A method of providing a sweeping motion to the distal portion of a flexible elongated instrument fitted with an inflatable actuation device capable of being actuated in different directions to unravel a bend in a tortuous passage, the method comprising:
providing the inflatable actuation device at a location along the length of the elongated instrument substantially proximal to a distal portion of the elongated instrument;
causing the distal portion of the elongated instrument to sweep in a predetermined direction by activating the inflatable actuation device by pressurizing the inflatable actuation device to apply a first force on the elongated instrument and bend the elongated instrument to a given direction; and
causing the distal portion of the elongated instrument to sweep in a direction opposite to the given direction by pressurizing the inflatable actuation device to applying a second force on the elongated instrument and bend the elongated device to the direction opposite to the given direction,
wherein the elongated instrument is bent and straightened repeatedly as the elongated instrument is made to advance into the tortuous passage.
